(19) United States
(12) Patent Application Publication
(10) Pub. No.: US 2006/0036313 A1
(43) Pub. Date: Feb. 16, 2006

(54) Apicoaortic conduit connector and method for using

(76) Inventor: Thomas A. Vassiliades, Atlanta, GA (US)

Correspondence Address: MORRISON & FOERSTER LLP, 1650 TYSONS BOULEVARD, SUITE 300, MCLEAN, VA, 22102, US

(21) Appl. No.: 10/915,691

(22) Filed: Aug. 11, 2004

(51) Int. Cl. A61F 2/06

(52) U.S. Cl. 623/1.23

(57) ABSTRACT

An improved apparatus and method for AAC insertion including a new AAC connector having a threaded or partially threaded body. The AAC connector has a flexible flange situated at one end that is soft and thin enough to bend backwards so that it can be pushed through an opening made in the apex, but rigid enough to flex back to its original position and hold its shape once it enters the interior of the left ventricle. A second ring is also provided that is adapted to be deployed over the body of the connector and against the exterior wall of the apex.

### FIELD OF INVENTION

This invention relates to devices and methods for creating an alternative conduit between the left ventricle and the aorta to create a double-outlet left ventricle.

### BACKGROUND

Construction of an alternative conduit between the left ventricle and the aorta (an apicoaortic conduit, or AAC) to create a double-outlet left ventricle (LV) has been successfully employed to treat a variety of complex congenital LV outflow obstruction (fibrous tunnel obstruction, aortic annular hypoplasia, tubular hypoplasia of the ascending aorta, and patients with diffuse septal thickening, severe LV hypertrophy and a small LV cavity) as well as adult-onset aortic stenosis in patients with complicating preoperative conditions (previous failed annular augmentation procedures, previous infection, previous CABG with patent anterior internal mammary artery grafts, and a porcelain ascending aorta).

However, the AAC insertion procedure has been poorly accepted, primarily because of early valve failures using first-generation bioprostheses as well as the success of direct LVOTO repair and aortic valve replacement. In the United States, despite an aging population, the unadjusted mortality for isolated aortic valve operations in 2001 remained under 4%. Further, the AAC insertion operation, with or without cardiopulmonary bypass, has not been as technically straightforward as direct aortic valve replacement. For most surgeons, AAC insertion is not a familiar operation and is of historical interest only.

Nonetheless, several studies have demonstrated that AAC insertion successfully lessens the LV-aortic pressure gradient, preserves or improves ventricular function and maintains normally distributed blood flow through the systemic and coronary circulation. While there have been several techniques described, the most commonly employed method is the lateral thoracotomy approach with placement of the AAC to the descending aorta. Other techniques include a median stemotomy approach with insertion of the distal limb of the AAC to the ascending aorta, to the transverse part of the aortic arch, or to the intra-abdominal supraceliac aorta.

In general, the thoracic aorta and the left ventricle apex are exposed through a left lateral thoracotomy, and a needle is passed through the apex and into the left ventricle. While the connector is still spaced apart from the apex, the sutures that will fix the connector to the apex are threaded through a cuff on the connector and through the apex in a matching pattern. The cuff is set back from the end of the connector by 1-2 centimeters to allow the end of the connector to extend through the heart muscle and into the left ventricle. Once the sutures are in place, a ventricular coring device is used to remove a core of ventricular muscle, and the pre-threaded sutures are then pulled to draw the connector into the opening until the cuff comes to rest on the apex. The sutures are tied off, and additional sutures may be added. Either before or after this procedure, the opposite end of the connector is attached to a valved conduit which terminates at the aorta.

The current techniques and technology available to perform AAC insertion were originally designed to be performed on-pump, either with an arrested or fibrillating heart. While off-pump cases have been described, they can be technically difficult.

### SUMMARY OF THE INVENTION

This invention describes an improved apparatus and method for AAC insertion that will significantly improve and simplify the insertion of a graft into the beating cardiac apex, making AAC insertion far more attractive. By creating a second outflow tract off pump, the detrimental effects of both CPB and global cardiac ischemia are avoided. Additionally, the conduction system is avoided as are the native coronary arteries and grafts from previous surgical revascularization. A small size valve (19 to 21 mm) for typical adult body surface areas is usually adequate, as the effective postoperative orifice is the sum of the native and prosthetic aortic valves. Further, valved conduit failure is far less likely with the availability of newer generation biologic valves.

According to the invention, a new AAC connector having a threaded or partially threaded body is provided. According to this embodiment, the AAC connector has a flexible flange situated at one end. The flexible flange is soft and thin enough to bend backwards so that it can be pushed through the opening in the apex, but rigid enough to flex back to its original position and hold its shape once it enters the interior of the left ventricle. The body of the graft is then drawn back so that the flexible flange presses against the inside wall of the left ventricle. This embodiment also includes a second ring adapted to be deployed over the body of the connector and against the exterior wall of the apex. Various means are described herein to secure the position of the second ring against the exterior wall of the apex so that no sutures are required.

According to one method for using the new AAC connector of the invention, a needle is passed through the apex and into the left ventricle. A guide wire is then inserted into the opening and, following dilation of the opening, an occlusion device is threaded over the wire and into the left ventricle and deployed. A ventricular coring device is then threaded in-line over the wire and a core of ventricular muscle is removed at the apex. while the occlusion device maintains hemostasis, the coring device is removed and the connector according to the invention is mounted on a dilator and introduced over the guide wire and occlusion device catheter. As the connector is introduced into the opening in the apex, the flange retracts. As the connector enters the left ventricle, it displaces the occlusion device to allow the flange to resume its normal shape. As discussed above, the connector is then drawn tight against the inside of the left ventricle and the second ring is deployed over the body of the connector to fit snugly against the apex. Once the connector is firmly in place, the occlusion device is withdrawn. The connector is then clamped shut while its free end is connected to the graft which terminates, or which will terminate, at the aorta.

Use of this new apparatus and method will significantly improve the ease and safety of AAC insertion. As persons of ordinary skill would readily appreciate, this method can also be used in a minimally invasive, endoscopically assisted approach.

### BRIEF DESCRIPTION OF THE DRAWINGS

The invention will be better understood by reference to the Detailed Description of the Invention when taken together with the attached drawings, wherein:

FIG. 1 is a perspective view of a first embodiment of an LV apical connector according to the invention;

FIG. 2 is a side view of the embodiment shown in FIG. 1;

FIG. 3 is a perspective view of a second embodiment of an LV apical connector according to the invention;

FIG. 4 is a side view of the embodiment shown in FIG. 3;

FIG. 5 is a side view of a third embodiment of an LV apical connector according to the invention.

### DETAILED DESCRIPTION OF THE INVENTION

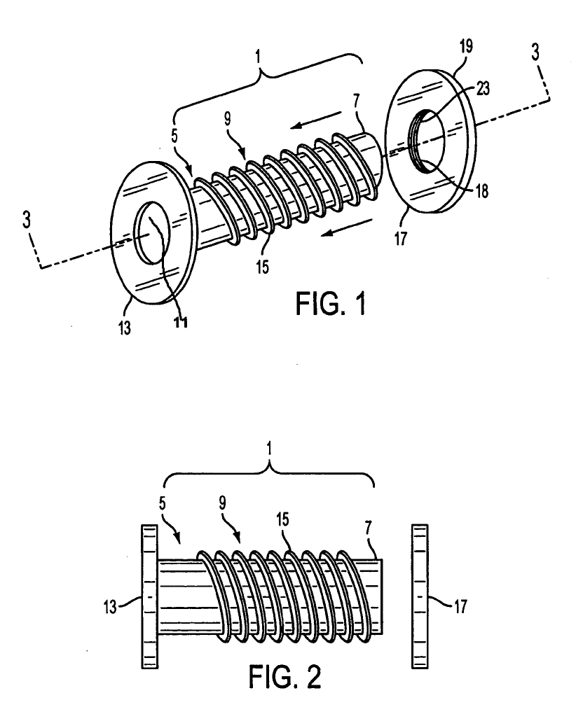

According to the embodiment shown in FIGS. 1 and 2, the connector according to the invention includes conduit 1 having an axis 3, distal end 5, a proximal end 7, an outer surface 9 and an inner surface 11. Distal end 5 is provided with a flange 13 extending from outer surface 9 in a direction away from axis 3. Flange 13 may be integrally formed with conduit 1, or it may be formed separately and permanently attached to distal end 5 of conduit 1 by known means. Conduit 1 should be sufficiently rigid to maintain its shape so as not to occlude the passage of blood therethrough during use. Flange 13 is sufficiently flexible to allow introduction of the distal end 5 into an opening having a diameter equal to or slightly less than the diameter of outer surface 9, but have sufficient stiffness and/or shape memory to flex back to its original position once it has passed through the opening.

Conduit 1 and flange 13 may be made of any suitable biocompatible material. Alternatively, conduit 1 and flange 13 may be coated with a biocompatible material. At least a portion of the outer surface 9 of conduit 1 may be threaded. Threading 15 may extend the entire length of conduit 1, or extend over only a portion thereof. In particular, threading 15 may be absent from a length of the distal end 5 of the conduit 1 that is slightly less than the thickness of the muscle at the apex. This alternative embodiment may serve to prevent over-tightening of the connector. According to another embodiment, threading 15 may not extend all the way to the proximal end 7.

External ring 17 has an inner diameter 18 and an outer diameter 19. Inner diameter 17 has threads 23 to correspond to the threading 15 on the outer surface 9 of conduit 1 The outer diameter 19 of external ring 17 may have any shape suitable to the designer, including circular or hexagonal. According to one embodiment of the invention, external ring 17 may be adapted to engage a tightening device (not shown) for tightening external ring 17 on conduit 1

External ring 17 may optionally be slightly convex, or have a convex surface facing flange 13 so as to better engage the heart muscle.

External ring may be made of any suitable biocompatible material. Alternatively, external ring 17 may be coated with a biocompatible material.

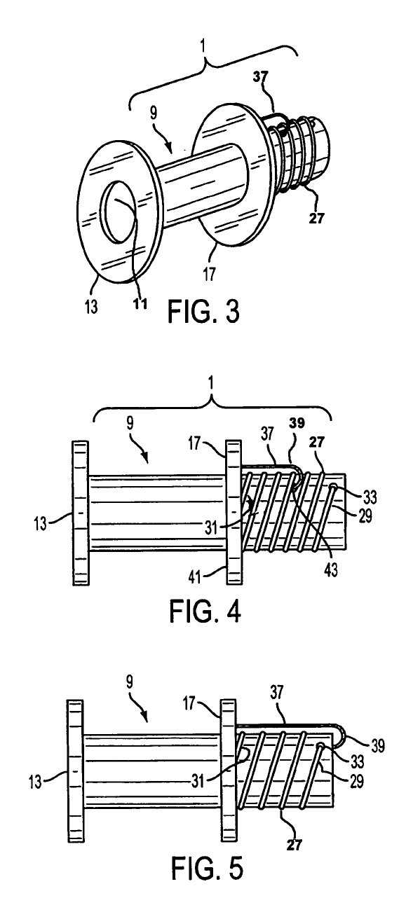

According to the embodiment shown in FIGS. 3 and 4, conduit 1 may be provided with an external ring 17 that is biased toward flange 13 by a biasing device 27, having proximal end 29 and distal end 31, that tends to force external ring 17 into contact with flange 13. As shown in FIGS. 4-6, the biasing device 27 may be a spring, in compression. As persons of ordinary skill in the art will appreciate, any biasing device may be used, including one or more flexible bands or rods. Conduit 1 may be provided with an engagement feature 33, such as a ring, slot or bore, to engage the proximal end 29 of the biasing device 27. Likewise, external ring 17 may be provided with an engagement feature (not shown) adapted to receive the distal end 31 of the biasing device 27.

Release device 37 may also be provided to releasably hold external ring 17 and biasing device 27 in pre-deployment configuration, with biasing device 27 in compression, until such a time as the flange 13 has been placed in the interior of the ventricle and the external ring 17 is ready to be deployed against the exterior surface of the heart muscle.

According to the embodiment shown in FIGS. 3 and 4, release device 37 may include one or more hooks 39 extending from the proximal surface 41 of the external ring 17 and adapted to releasably engage an engagement feature 43, for example, a slot or bore, in conduit 1. Alternatively, as shown in FIG. 5, the release device 37 may extend to and hook over the proximal end 7 of conduit 1.

A portion of conduit 1 optionally may be threaded and the inside diameter of external ring threaded to permit further tightening of external ring on conduit 1 after deployment of the external ring following removal of the release device.

In accordance with one method for using the connector of the invention, a needle is passed through the apex and into the left ventricle. A guide wire is then inserted into the opening and, following dilation of the opening, an occlusion device is threaded over the wire and into the left ventricle and deployed. The occlusion device may include known occlusion devices such as an occlusion balloon, the Guidant Heartstring™ disclosed at http://www.guidant.com/products/producttemplates/cs/heartstring.shtml, or the Baladi inverter, disclosed in U.S. Pat. Nos. 5,944,730 and 6,409,739. A ventricular coring device is then threaded in-line over the occlusion device and a core of ventricular muscle is removed at the apex. In addition to known coring techniques, an annular contact laser may be used to vaporize the tissue along the perimeter of the core. The cored tissue may then be removed according to known methods. According to a further alternative embodiment, a contact laser may be used to vaporize the entire area of the core, eliminating the need to remove cored tissue. No matter the method of coring, once coring has been completed, the coring device is removed while the occlusion device maintains hemostasis, and the connector according to the invention is mounted on a dilator and introduced over the guide wire and occlusion device. As the connector is introduced into the opening in the apex, the flange 13 retracts. As the connector enters the left ventricle, it displaces the occlusion device to allow the flange 13 to resume its normal shape. As discussed above, the connector is then drawn tight against the inside of the left ventricle. According to a first embodiment, external ring 17 is threaded onto conduit 1 and tightened until it is snug against the exterior wall of the apex.

According to a second embodiment, once the flange has been introduced into the ventricle and pulled back to engage the interior ventricle wall, release device 37 is released, allowing biasing device 27 to force the external ring 17 against the external wall of the apex. According to a further aspect of this embodiment of the invention, threads on the inside diameter of external ring may be made to engage threads oh the outer surface of conduit 1 to further secure external ring against the wall of the apex.

Once the connector is firmly in place, the occlusion device is withdrawn. The connector is then clamped shut while its free end is connected to the graft which terminates, or which will terminate, at the aorta.

We claim:

1. An apicoaortic conduit connector comprising:
a tube having proximal and distal ends, and inside and outside surfaces;
a flexible flange disposed at or near said distal end;
a securing ring adapted to fit over said tube.

2. An apicoaortic conduit connector according to claim 1 further comprising threading on at least a portion of said outside surface of said tube and corresponding threading on an inside diameter of said securing ring.

3. An apicoaortic conduit connector according to claim 1 wherein said flexible flange is sufficiently flexible to deform sufficiently to allow the proximal end of said connector, together with said flange, to be inserted through an opening in a left ventricle, said opening having a diameter equal to or less than the diameter of said tube.

4. An apicoaortic conduit connector according to claim 3 wherein said flexible flange has sufficient shape memory that once it has been deformed to allow insertion of the proximal end of said connector into said opening in a left ventricle, it will return to its original shape.

5. An apicoaortic conduit connector according to claim 1, further comprising a biasing device attached at one end to said tube, and attached at another end to a proximal surface of said securing ring.

6. An apicoaortic conduit connector according to claim 5, further comprising a release device attached at one end to said securing ring, and releasably attached at another end to said tube, and releasably holding said biasing device in compression.
